(19) United States
(12) Patent Application Publication
(10) Pub. No.: US 2011/0010985 A1
(43) Pub. Date: Jan. 20, 2011

(54) Fuel Additive to Control Deposit Formation

(76) Inventors: Peter Wangqi Hou, Chesterfield, VA (US); William J. Colucci, Glen Allen, VA (US); Thomas William Nichols, Richmond, VA (US)

Correspondence Address: Mannava & Kang, P.C., 11240 Waples Mill Road, Suite 300, Fairfax, VA, 22030, US

(21) Appl. No.: 12/886,841

(22) Filed: Sep. 21, 2010

Related U.S. Application Data
(62) Division of application No. 11/752,068, filed on May 22, 2007.

(51) Int. Cl. C07D 207/40; C10L 1/232

(52) U.S. Cl. 44/347; 548/545

(57) ABSTRACT

The present disclosure is directed to a fuel composition comprising a deposit-modifying effective amount of a hydrocarbyl-substituted succinimide; a detergent; and a fuel. A method for reducing deposit formation is also disclosed.

This application is a divisional application of U.S. patent application Ser. No. 11/752,068, filed May 22, 2007, the entire disclosure of which is hereby incorporated by reference.

### FIELD OF THE DISCLOSURE

The present disclosure relates to a fuel composition comprising a deposit-modifying effective amount of a hydrocarbyl-substituted succinimide; a detergent; and a fuel. A method for reducing deposit formation is also disclosed.

### BACKGROUND OF THE DISCLOSURE

Over the years considerable work has been devoted to additives for controlling (preventing or reducing) deposit formation in the fuel induction systems of spark-ignition internal combustion engines. In particular, additives that can effectively control fuel injector deposits, intake valve deposits and combustion chamber deposits represent the focal point of considerable research activities in the field and despite these efforts, further improvements are desired.

A variety of systems have been devised to supply fine liquid fuel droplets and air to internal combustion engines. These systems either supply fuel directly into the combustion chamber (direct injection) or utilize a carburetor or fuel injector(s) to supply the mixture through an intake manifold into a combustion chamber (indirect injection). In currently employed systems, the fuel-air mixture is produced by atomizing a liquid fuel and supplying it as fine droplets into an air stream.

In conventional spark-ignited engines employing port-fuel injection, the injected fuel is vaporized by directing the liquid fuel droplets at hot components in the intake port or manifold, under normal operating conditions. The liquid fuel films on the surfaces of the hot components and is subsequently vaporized. The mixture of vaporized fuel and intake air is then drawn into the cylinder by the pressure differential created as the intake valve opens and the piston moves towards bottom dead center. To ensure a degree of control that is compatible with modern engines, this vaporizing technique is typically optimized to occur in less than one engine cycle.

Under most engine operating conditions, the temperature of the intake components is sufficient to rapidly vaporize the impinging liquid fuel droplets. However, under conditions such as cold-start and warm-up, the fuel is not vaporized through impingement on the relatively cold engine components. Instead, engine operation under these conditions is ensured by supplying excess fuel such that a sufficient fraction evaporates through heat and mass transfer as it travels through the air prior to impinging on a cold intake component. Evaporation rate through this mechanism is a function of fuel properties, temperature, pressure, relative droplet and air velocities and droplet diameter. Of course, this approach breaks down in extreme ambient cold-starts, in which the fuel volatility is insufficient to produce vapor in ignitable concentrations with air.

In order for combustion to be chemically complete, the fuel-air mixture must be vaporized to a stoichiometric gas-phase mixture. A stoichiometric combustible mixture contains the exact quantities of air (oxygen) and fuel required for complete combustion. For gasoline, this air-fuel ratio is about 14.7:1 by weight. A fuel-air mixture that is not completely vaporized, nor stoichiometric, results in incomplete combustion and reduced thermal efficiency. The products of an ideal combustion process are water (H₂O) and carbon dioxide (CO₂). If combustion is incomplete, some carbon is not fully oxidized, yielding carbon monoxide (CO) and unburned hydrocarbons (HC). This leads to engine deposits and injector fouling.

There is a desire in the petroleum industry to produce a fuel having effective injector deposit control. EP 0 020 037 discloses that the use of an oil-soluble, C₁₂₋₃₆ aliphatic hydrocarbyl succinimide or succinimide provides a friction reducing effect when it is incorporated into a lubricating oil, such as for use in a crankcase. The hydrocarbyl succinic anhydride is reacted with ammonia to form the succinimide. The reference discloses that the succinimide can also be used in both diesel fuel and gasoline. However, the reference does not teach that the succinimide can be used in fuel composition in a deposit-modifying effective amount. In fact, the reference is silent with respect to the problem of deposit formation in port-fuel injector engines.

### SUMMARY OF THE DISCLOSURE

In an aspect, there is disclosed a fuel composition comprising a deposit-modifying effective amount of a hydrocarbyl-substituted succinimide; a detergent; and a fuel.

There is also disclosed a method for reducing deposit formation in an engine comprising providing to the engine a fuel composition comprising a deposit-modifying effective amount of a hydrocarbyl-substituted succinimide; a detergent; and a fuel.

Additional objects and advantages of the disclosure will be set forth in part in the description which follows, and/or can be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### DESCRIPTION OF THE EMBODIMENTS

As used herein the term “succinimide” is meant to encompass the completed reaction product from reaction between ammonia and a hydrocarbyl-substituted succinic acid or anhydride (or like succinic acylating agent), and is intended to encompass compounds wherein the product may have amide, and/or salt linkages in addition to the imide linkage of the type that results from the reaction of or contact with ammonia, and an anhydride moiety. By “reacting” herein with regard to the alkylation is meant the product or result of contacting, exposing or bringing together any of the recited components or chemicals, whether a covalent bond, ionic bond, salt or other association is produced.

The hydrocarbyl-substituted succinimides of the fuels of this disclosure are well known. They are readily made by first reacting an olefinically unsaturated hydrocarbon of a desired molecular weight with maleic anhydride to form a hydrocarbyl-substituted succinic anhydride. Reaction temperatures of about 100° C. to about 250° C. can be used. With higher boiling olefinically-unsaturated hydrocarbons, good results are obtained at about 200° C. to about 250° C. This reaction can be promoted by the addition of chlorine. Alkenyl succinimides in which the succinic group contains a hydrocarbyl substituent containing at least 40 carbon atoms are described for example in U.S. Pat. Nos. 3,172,892; 3,202,678; 3,216,936; 3,219,666; 3,254,025; 3,272,746; 4,234,435; 4,613,341; and 5,575,823, the disclosures of all of which are hereby incorporated by reference.

Typical olefins include, but are not limited to, cracked wax olefins, linear alpha olefins, branched chain alpha olefins, polymers and copolymers of lower olefins. The olefins can be chosen from ethylene, propylene, butylene, such as isobutylene, 1-octane, 1-hexene, 1-decene and the like. Useful polymers and/or copolymers include, but are not limited to, polypropylene, polybutenes, polyisobutene, ethylene-propylene copolymers, ethylene-isobutylene copolymers, propylene-isobutylene copolymers, ethylene-1-decene copolymers and the like.

Hydrocarbyl substituents have also been made from olefin terpolymers. Very useful products can be made from ethylene-C₃₋₁₂ alpha olefin-C₅₋₁₂ non-conjugated diene terpolymers; such as ethylene-propylene-1,4-hexadiene terpolymer; ethylenepropylene-1,5-cyclooctadiene terpolymer; ethylene-propylenenorbornene terpolymers and the like.

In one embodiment, the hydrocarbyl substituents are derived from butene polymers, for example polymers of isobutylene. Suitable polyisobutenes for use in preparing the succinimide-acids of the present disclosure can in one embodiment include those polyisobutenes that comprise at least about 20% of the more reactive methylvinylidene isomer, for example at least 50%, and as a further example at least 70%. Suitable polyisobutenes include those prepared using BF₃ catalysts. The preparation of such polyisobutenes in which the methylvinylidene isomer comprises a high percentage of the total composition is described in U.S. Pat. Nos. 4,152,499 and 4,605,808, the disclosures of which are hereby incorporated by reference.

The molecular weight of the hydrocarbyl substituent can vary over a wide range. The hydrocarbyl group can have a molecular weight of less than 600. An exemplary range is about 100 to about 300 number average molecular weight, for example from about 150 to about 275, as determined by gel permeation chromatography (GPC). In an aspect, the number average molecular weight of the hydrocarbyl group is less than about 350. Thus, hydrocarbyl groups of predominantly C₄-C₃₆ are useful herein with C₁₄-C₁₈ hydrocarbyl groups being particularly effective on the succinimide in providing improved lubricity to the low sulfur middle distillate fuel. In an aspect, hydrocarbyl groups of up to about C₂₄ are also useful.

Carboxylic reactants other than maleic anhydride can be employed such as maleic acid, fumaric acid, malic acid, tartaric acid, itaconic acid, itaconic anhydride, citraconic acid, citraconic anhydride, mesaconic acid, ethylmaleic anhydride, dimethylmaleic anhydride, ethylmaleic acid, dimethylmaleic acid, hexylmaleic acid, and the like, including the corresponding acid halides and lower aliphatic esters.

For example, hydrocarbyl-substituted succinic anhydrides may be prepared by the thermal reaction of a polyolefin and maleic anhydride, as described, for example in U.S. Pat. Nos. 3,361,673 and 3,676,089, the disclosures of which are incorporated by reference. Alternatively, the substituted succinic anhydrides can be prepared by the reaction of chlorinated polyolefins with maleic anhydride, as described, for example, in U.S. Pat. No. 3,172,892, the disclosure of which is incorporated by reference. A further discussion of hydrocarbyl-substituted succinic anhydrides can be found, for example, in U.S. Pat. Nos. 4,234,435; 5,620,486 and 5,393,309, the disclosures of which are incorporated by reference.

The mole ratio of maleic anhydride to olefin unsaturated hydrocarbon can vary widely. It can vary from about 5:1 to about 1:5, for example from about 3:1 to about 1:3, and as a further example the maleic anhydride can be used in stoichiometric excess to force the reaction to completion. The unreacted maleic anhydride can be removed by vacuum distillation.

The reaction between the hydrocarbyl-substituted succinic anhydride and the ammonia can in one embodiment be carried out by mixing the components and heating the mixture to a temperature high enough to cause a reaction to occur but not so high as to cause decomposition of the reactants or products or the anhydride may be heated to reaction temperature and the ammonia added over an extended period. A useful temperature is about 100° C. to about 250° C. Exemplary results can be obtained by conducting the reaction at a temperature high enough to distill out water formed in the reaction.

The hydrocarbyl-substituted succinimide can be present in the fuel composition in any desired or effective amount. In an aspect, the hydrocarbyl-substituted succinimide can be present in an amount ranging from about 1 ppm to about 1,000 ppm, for example from about 3 ppm to about 25 ppm, and as a further example from about 5 ppm to about 10 ppm by weight, relative to the total weight of the fuel composition.

The disclosed fuel composition can also optionally comprise a detergent/dispersant, such as a Mannich base detergent, a polyetheramines, and/or a polyisobutylene amine. The Mannich base detergents suitable for use in the present disclosure include the reaction products of a high molecular weight alkyl-substituted hydroxyaromatic compound, aldehydes and amines. The alkyl-substituted hydroxyaromatic compound, aldehydes and amines used in making the Mannich reaction products of the present disclosure can be any such compounds known and applied in the art, in accordance with the foregoing limitations.

The high molecular weight alkyl substituents on the benzene ring of the hydroxyaromatic compound can be derived from a polyolefin having a number average molecular weight (Mn) of from about 500 to about 3000, and for example from about 700 to about 2100, as determined by gel permeation chromatography (GPC). The polyolefin used can have a polydispersity (weight average molecular weight/number average molecular weight) in the range of about 1 to about 4 (for example from about 1 to about 2) as determined by GPC.

The alkylation of the hydroxyaromatic compound can be typically performed in the presence of an alkylating catalyst at a temperature in the range of about 0 to about 200° C., for example 0 to 100° C. Acidic catalysts can be generally used to promote Friedel-Crafts alkylation. Typical catalysts used in commercial production include sulphuric acid, BF₃, aluminum phenoxide, methanesulphonic acid, cationic exchange resin, acidic clays and modified zeolites.

Polyolefins suitable for forming the high molecular weight alkyl-substituted hydroxyaromatic compounds include polypropylene, polybutenes, polyisobutylene, copolymers of butylene and/or butylene and propylene, copolymers of butylene and/or isobutylene and/or propylene, and one or more mono-olefinic comonomers copolymerizable therewith (e.g., ethylene, 1-pentene, 1-hexene, 1-octene, 1-decene, etc.) where the copolymer molecule contains at least 50% by weight, of butylene and/or isobutylene and/or propylene units. The comonomers polymerized with propylene or such butenes can be aliphatic and can also contain non-aliphatic groups, e.g., styrene, o-methylstyrene, p-methylstyrene, divinyl benzene and the like. Thus in any case the resulting polymers and copolymers used in forming the high molecular weight alkyl-substituted hydroxyaromatic compounds can be substantially aliphatic hydrocarbon polymers.

Polybutylene is preferred. Unless otherwise specified herein, the term “polybutylene” is used in a generic sense to include polymers made from “pure” or “substantially pure” 1-butene or isobutene, and polymers made from mixtures of two or all three of 1-butene, 2-butene and isobutene. Commercial grades of such polymers can also contain insignificant amounts of other olefins. So-called high reactivity polyisobutenes having relatively high proportions of polymer molecules having a terminal vinylidene group can also be suitable for use in forming the long chain alkylated phenol reactant. Suitable high-reactivity polyisobutenes include those polyisobutenes that comprise at least about 20% of the more reactive methylvinylidene isomer, for example at least 50% and as a further example at least 70%. Suitable polyisobutenes include those prepared using BF₃ catalysts. The preparation of such polyisobutenes in which the methylvinylidene isomer comprises a high percentage of the total composition is described in U.S. Pat. Nos. 4,152,499 and 4,605,808.

The Mannich detergent can be made from a high molecular weight alkylphenol or alkylcresol. However, other phenolic compounds can be used including high molecular weight alkyl-substituted derivatives of resorcinol, hydroquinone, catechol, hydroxydiphenyl, benzylphenol, phenethylphenol, naphthol, tolylnaphthol, among others. Preferred for the preparation of the Mannich detergents are the polyalkylphenol and polyalkylcresol reactants, e.g., polypropylphenol, polybutylphenol, polypropylcresol and polybutylcresol, wherein the alkyl group has a number average molecular weight of about 500 to about 2100, while the most preferred alkyl group is a polybutyl group derived from polyisobutylene having a number average molecular weight in the range of about 700 to about 1300.

An exemplary configuration of the high molecular weight alkyl-substituted hydroxyaromatic compound can be that of a para-substituted mono-alkylphenol or a para-substituted mono-alkyl ortho-cresol. However, any hydroxyaromatic compound readily reactive in the Mannich condensation reaction can be employed. Thus, Mannich products made from hydroxyaromatic compounds having only one ring alkyl substituent, or two or more ring alkyl substituents can be suitable for use in this disclosure. The long chain alkyl substituents can contain some residual unsaturation, but in general, can be substantially saturated alkyl groups.

Representative amine reactants include, but are not limited to, alkylene polyamines having at least one suitably reactive primary or secondary amino group in the molecule. Other substituents such as hydroxyl, cyano, amido, etc., can be present in the polyamine. In an aspect, the alkylene polyamine can be a polyethylene polyamine. Suitable alkylene polyamine reactants include, but are not limited to, ethylenediamine, diethylenetriamine, triethylenetetramine, tetraethylenepentamine and mixtures of such amines having nitrogen contents corresponding to alkylene polyamines of the formula H₂N-(A-NH—)ₙH, where A can be divalent ethylene or propylene and n can be an integer of from 1 to 10, for example 1 to 4. The alkylene polyamines can be obtained by the reaction of ammonia and dihalo alkanes, such as dichloro alkanes.

The amine can also be an aliphatic diamine having one primary or secondary amino group and at least one tertiary amino group in the molecule. Examples of suitable polyamines include, but are not limited to, N,N,N″,N″-tetraalkyldialkylenetriamines (two terminal tertiary amino groups and one central secondary amino group), N,N,N′,N″-tetraalkyltrialkylenetetramines (one terminal tertiary amino group, two internal tertiary amino groups and one terminal primary amino group), N,N,N′,N″,N′″-pentaalkyltrialkylenetetramines (one terminal tertiary amino group, two internal tertiary amino groups and one terminal secondary amino group), N,N-dihydroxyalkyl-alpha, omega-alkylenediamines (one terminal tertiary amino group and one terminal primary amino group), N,N,N′-trihydroxyalkyl-alpha, omega-alkylenediamines (one terminal tertiary amino group and one terminal secondary amino group), tris(dialkylaminoalkyl)aminoalkylmethanes (three terminal tertiary amino groups and one terminal primary amino group), and similar compounds, wherein the alkyl groups are the same or different and typically contain no more than about 12 carbon atoms each, and which for example contain from 1 to 4 carbon atoms each. In an aspect, these alkyl groups can be methyl and/or ethyl groups. In another aspect, polyamine reactants can be N,N-dialkyl-alpha, omega-alkylenediamine, such as those having from 3 to about 6 carbon atoms in the alkylene group and from 1 to about 12 carbon atoms in each of the alkyl groups, which for example can be the same but which can be different. In an aspect, N,N-dimethyl-1,3-propanediamine and N-methyl piperazine can be used.

Examples of polyamines having one reactive primary or secondary amino group that can participate in the Mannich condensation reaction, and at least one sterically hindered amino group that cannot participate directly in the Mannich condensation reaction to any appreciable extent include N-(tert-butyl)-1,3-propanediamine, N-neopentyl-1,3-propanediamine, N-(tert-butyl)-1-methyl-1,2-ethanediamine, N-(tert-butyl)-1-methyl-1,3-propanediamine, and 3,5-di(tert-butyl)aminoethylpiperazine.

Representative aldehydes for use in the preparation of the Mannich base products include the aliphatic aldehydes such as formaldehyde, acetaldehyde, propionaldehyde, butyraldehyde, valeraldehyde, caproaldehyde, heptaldehyde, stearaldehyde. Aromatic aldehydes which can be used include, but are not limited to, benzaldehyde and salicylaldehyde. Illustrative heterocyclic aldehydes for use herein are furfural and thiophene aldehyde, etc. Also useful are formaldehyde-producing reagents such as paraformaldehyde, or aqueous formaldehyde solutions such as formalin. As an example, formaldehyde or formalin can be used.

The condensation reaction among the alkylphenol, the specified amine(s) and the aldehyde can be conducted at a temperature typically in the range of about 40° to about 200° C. The reaction can be conducted in bulk (no diluent or solvent) or in a solvent or diluent. Water is evolved and can be removed by azeotropic distillation during the course of the reaction. Typically, the Mannich reaction products can be formed by reacting the alkyl-substituted hydroxyaromatic compound, the amine and aldehyde in the molar ratio of 1.0:0.5-2.0:1.0-3.0, respectively.

Suitable Mannich base detergents for use in the present disclosure include those detergents taught in U.S. Pat. Nos. 4,231,759; 5,514,190; 5,634,951; 5,697,988; 5,725,612; and 5,876,468, the disclosures of which are incorporated herein by reference.

Hydrocarbyl amine detergents are known materials prepared by known process technology. One common process involves halogenation of a long chain aliphatic hydrocarbon such as a polymer of ethylene, propylene, butylene, isobutene, or copolymers such as ethylene and propylene, butylene and isobutylene, and the like, followed by reaction of the resultant halogenated hydrocarbon with a polyamine. If desired, at least some of the product can be converted into an amine salt by treatment with an appropriate quantity of an acid. The products formed by the halogenation route often contain a small amount of residual halogen such as chlorine. Another way of producing suitable aliphatic polyamines involves controlled oxidation (e.g., with air or a peroxide) of a polyolefin such as polyisobutene followed by reaction of the oxidized polyolefin with a polyamine. For synthesis details for preparing such aliphatic polyamine detergent/dispersants, see for example U.S. Pat. Nos. 3,438,757; 3,454,555; 3,485,601; 3,565,804; 3,573,010; 3,574,576; 3,671,511; 3,746,520; 3,756,793; 3,844,958; 3,852,258; 3,864,098; 3,876,704; 3,884,647; 3,898,056; 3,950,426; 3,960,515; 4,022,589; 4,039,300; 4,128,403; 4,166,726; 4,168,242; 5,034,471; 5,086,115; 5,112,364; and 5,124,484; and published European Patent Application 384,086. The disclosures of each of the foregoing documents are incorporated herein by reference. The long chain substituent(s) of the hydrocarbyl amine detergent can contain(s) an average of 50 to 350 carbon atoms in the form of alkyl or alkenyl groups (with or without a small residual amount of halogen substitution). Alkenyl substituents derived from poly-alpha-olefin homopolymers or copolymers of appropriate molecular weight (e.g., propene homopolymers, butene homopolymers, C₃ and C₄ alpha-olefin copolymers, and the like) can be suitable. For example, the substituent can be a polyisobutenyl group formed from polyisobutene having a number average molecular weight (as determined by gel permeation chromatography) in the range of about 500 to about 2000, for example about 600 to about 1800, and as a further example about 700 to about 1600.

Polyetheramines suitable for use as the detergents of the present disclosure can be “single molecule” additives, incorporating both amine and polyether functionalities within the same molecule. The polyether backbone can be based on propylene oxide, ethylene oxide, butylene oxide, or mixtures of these. Exemplary ones include propylene oxide or butylene oxide or mixture thereof to impart good fuel solubility. The polyetheramines can be monoamines, diamines or triamines. The molecular weight of the polyetheramines will typically range from 500 to 3000. Other suitable polyetheramines are those compounds taught in U.S. Pat. Nos. 4,191,537; 4,236,020; 4,288,612; 5,089,029; 5,112,364; 5,322,529; 5,514,190 and 5,522,906, the disclosures of which are incorporated by reference herein.

The detergent for use in the disclosed fuel composition can be present in any desired or effective amount. For example, the detergent can be present in an amount of from about 10 ppm to about 1500 ppm, and as a further example from about 100 ppm to about 300 ppm, by weight, relative to the total weight of the fuel composition.

The base fuels used in formulating the fuel compositions of the present disclosure include any base fuels suitable for use in the operation of direct injection gasoline engines such as leaded or unleaded motor gasolines, and so-called reformulated gasolines. The fuel can be a mixture of hydrocarbons boiling in the gasoline boiling range. This fuel may comprise straight chain or branch chain paraffins, cycloparaffins, olefins, aromatic hydrocarbons or any mixture of these. The gasoline can be derived from straight run naptha, polymer gasoline, natural gasoline or from catalytically reformed stocks boiling in the range from about 80° to about 450° F. The octane level of the gasoline is not critical and any conventional gasoline can be employed.

By “fuel” herein is meant hydrocarbonaceous fuels such as, but not limited to, diesel fuel, jet fuel, alcohols, ethers, kerosene, low sulfur fuels, synthetic fuels, such as Fischer-Tropsch fuels, liquid petroleum gas, bunker oils, gas to liquid (GTL) fuels, coal to liquid (CTL) fuels, biomass to liquid (BTL) fuels, high asphaltene fuels, fuels derived from coal (natural, cleaned, and petcoke), genetically engineered biofuels and crops and extracts therefrom, natural gas, propane, butane, unleaded motor and aviation gasolines, and so-called reformulated gasolines which typically contain both hydrocarbons of the gasoline boiling range and fuel-soluble oxygenated blending agents, such as alcohols, ethers and other suitable oxygen-containing organic compounds. Oxygenates suitable for use in the fuels of the present disclosure include methanol, ethanol, isopropanol, t-butanol, mixed alcohols, methyl tertiary butyl ether, tertiary amyl methyl ether, ethyl tertiary butyl ether and mixed ethers. Oxygenates, when used, will normally be present in the reformulated gasoline fuel in an amount below about 25% by volume, and for example in an amount that provides an oxygen content in the overall fuel in the range of about 0.5 to about 5 percent by volume. “Hydrocarbonaceous fuel” or “fuel” herein shall also mean waste or used engine or motor oils which may or may not contain molybdenum, gasoline, bunker fuel, coal (dust or slurry), crude oil, refinery “bottoms” and by-products, crude oil extracts, hazardous wastes, yard trimmings and waste, wood chips and saw dust, agricultural waste, fodder, silage, plastics and other organic waste and/or by-products, and mixtures thereof, and emulsions, suspensions, and dispersions thereof in water, alcohol, or other carrier fluids. By “diesel fuel” herein is meant one or more fuels selected from the group consisting of diesel fuel, biodiesel, biodiesel-derived fuel, synthetic diesel and mixtures thereof. In an aspect, the hydrocarbonaceous fuel is substantially sulfur-free, by which is meant a sulfur content not to exceed on average about 30 ppm of the fuel.

In an aspect, the detergents can be used with a liquid carrier or induction aid. Such carriers can be of various types, such as for example liquid poly-.alpha.-olefin oligomers, mineral oils, liquid poly(oxyalkylene) compounds, liquid alcohols or polyols, polyalkenes, liquid esters, and similar liquid carriers. Mixtures of two or more such carriers can be employed.

Exemplary liquid carriers include 1) a mineral oil or a blend of mineral oils that have a viscosity index of less than about 120, 2) one or more poly-.alpha.-olefin oligomers, 3) one or more poly(oxyalkylene) compounds having an average molecular weight in the range of about 500 to about 3000, 4) polyalkenes, 5) polyalkyl-substituted hydroxyaromatic compounds or 6) mixtures thereof. The mineral oil carrier fluids that can be used include paraffinic, naphthenic and asphaltic oils, and can be derived from various petroleum crude oils and processed in any suitable manner. For example, the mineral oils can be solvent extracted or hydrotreated oils. Reclaimed mineral oils can also be used. Hydrotreated oils can also be used. For example, the mineral oil used can have a viscosity at 40° C. of less than about 1600 SUS, and for example range from about 300 to about 1500 SUS at 40° C. Paraffinic mineral oils can have viscosities at 40° C. in the range of about 475 SUS to about 700 SUS. For best results, it can be highly desirable that the mineral oil have a viscosity index of less than about 100, for example, less than about 70, and as a further example in the range of from about 30 to about 60.

The poly-.alpha.-olefins (PAO) suitable for use as carrier fluids are the hydrotreated and unhydrotreated poly-alpha-olefin oligomers, i.e., hydrogenated or unhydrogenated products, primarily trimers, tetramers and pentamers of alpha-olefin monomers, which monomers contain from 6 to 12, generally 8 to 12, and for example about 10 carbon atoms. Their synthesis is outlined in U.S. Pat. Nos. 3,763,244; 3,780,128; 4,172,855; 4,218,330; and 4,950,822, the disclosures of which are hereby incorporated by reference. The usual process essentially comprises catalytic oligomerization of short chain linear alpha olefins (suitably obtained by catalytic treatment of ethylene). The poly-alpha-olefins used as carriers will usually have a viscosity (measured at 100° C.) in the range of 2 to 20 centistokes (cSt). In an aspect, the poly-alpha-olefin has a viscosity of at least 8 cSt, and for example about 10 cSt at 100° C.

The poly(oxyalkylene) compounds which are among the exemplary carrier fluids for use herein can be fuel-soluble compounds which can be represented by the following formula R_{A}—(R_{B}—O)ₙ—R_{C} wherein R_{A} is typically a hydrogen, alkoxy, cycloalkoxy, hydroxy, amino, hydrocarbyl (e.g., alkyl, cycloalkyl, aryl, alkylaryl, aralkyl, etc.), amino-substituted hydrocarbyl, or hydroxy-substituted hydrocarbyl group, R_{B} is an alkylene group having 2-10 carbon atoms (for example 2-4 carbon atoms), R_{C} is typically a hydrogen, alkoxy, cycloalkoxy, hydroxy, amino, hydrocarbyl (e.g., alkyl, cycloalkyl, aryl, alkylaryl, aralkyl, etc.), amino-substituted hydrocarbyl, or hydroxy-substituted hydrocarbyl group, and n is an integer from 1 to 500 and for example in the range of from 3 to 120 representing the number (usually an average number) of repeating alkyleneoxy groups. In compounds having multiple —R_{B}—O— groups, R_{B} can be the same or different alkylene group and where different, can be arranged randomly or in blocks. Exemplary poly(oxyalkylene) compounds are monools comprised of repeating units formed by reacting an alcohol with one or more alkylene oxides, for example one alkylene oxide, and as a further example propylene oxide or butylene oxide.

The average molecular weight of the poly(oxyalkylene) compounds used as carrier fluids can be in the range of from about 500 to about 3000, for example from about 750 to about 2500, and as a further example from above about 1000 to about 2000.

One useful sub-group of poly(oxyalkylene) compounds is comprised of the hydrocarbyl-terminated poly(oxyalkylene) monools such as are referred to in the passage at column 6, line 20 to column 7 line 14 of U.S. Pat. No. 4,877,416 and references cited in that passage, said passage and said references being fully incorporated herein by reference.

An exemplary sub-group of poly(oxyalkylene) compounds is comprised of one or a mixture of alkylpoly(oxyalkylene)monools which in its undiluted state is a gasoline-soluble liquid having a viscosity of at least about 70 centistokes (cSt) at 40° C. and at least about 13 cSt at 100° C. Of these compounds, monools formed by propoxylation of one or a mixture of alkanols having at least about 8 carbon atoms, and for example in the range of about 10 to about 18 carbon atoms, can be used.

The poly(oxyalkylene) carriers used herein can have viscosities in their undiluted state of at least about 60 cSt at 40° C. (for example at least about 70 cSt at 40° C.) and at least about 11 cSt at 100° C. (for example at least about 13 cSt at 100° C.). In addition, the poly(oxyalkylene) compounds used herein for example can have viscosities in their undiluted state of no more than about 400 cSt at 40° C. and no more than about 50 cSt at 100° C. For example, their viscosities will not exceed about 300 cSt at 40° C. and will not exceed about 40 cSt at 100° C.

Exemplary poly(oxyalkylene) compounds can also include poly(oxyalkylene) glycol compounds and monoether derivatives thereof that satisfy the above viscosity requirements and that are comprised of repeating units formed by reacting an alcohol or polyalcohol with an alkylene oxide, such as propylene oxide and/or butylene oxide with or without use of ethylene oxide, and especially products in which at least 80 mole % of the oxyalkylene groups in the molecule are derived from 1,2-propylene oxide. Details concerning preparation of such poly(oxyalkylene) compounds are referred to, for example, in U.S. Pat. Nos. 2,425,755; 2,425,845; 2,448,664; and 2,457,139, and are fully incorporated herein by reference.

The poly(oxyalkylene) compounds, when used, can contain a sufficient number of branched oxyalkylene units (e.g., methyldimethyleneoxy units and/or ethyldimethyleneoxy units) to render the poly(oxyalkylene) compound gasoline soluble.

Suitable poly(oxyalkylene) compounds for use herein include those taught in U.S. Pat. Nos. 5,514,190; 5,634,951; 5,697,988; 5,725,612; 5,814,111 and 5,873,917, the disclosures of which are incorporated herein by reference.

The polyalkenes suitable for use as carrier fluids herein include polypropene and polybutene. The polyalkenes of the present disclosure can have a molecular weight distribution (Mw/Mn) of less than 4. In an aspect, the polyalkenes have a MWD of 1.4 or below. Preferred polybutenes have a number average molecular weight (Mn) of from about 500 to about 2000, for example about 600 to about 1000, as determined by gel permeation chromatography (GPC). Suitable polyalkenes for use in the present invention are taught in U.S. Pat. No. 6,048,373, the disclosure of which is hereby incorporated by reference.

The polyalkyl-substituted hydroxyaromatic compounds suitable for use as carrier fluid herein can include those compounds known in the art as taught in U.S. Pat. Nos. 3,849,085; 4,231,759; 4,238,628; 5,300,701; 5,755,835 and 5,873,917, the disclosures of which are incorporated herein by reference.

The fuel compositions of the present disclosure can contain other additives. Non-limiting examples of additives include dispersants/detergents, antioxidants, thermal stabilizers, carrier fluids, metal deactivators, dyes, markers, corrosion inhibitors, biocides, antistatic additives, drag reducing agents, demulsifiers, emulsifiers, dehazers, anti-icing additives, antiknock additives, anti-valve-seat recession additives, surfactants, other lubricity additives, combustion improvers, cetane number improvers and mixtures thereof.

The disclosed fuel composition can be combusted in any combustion system, including but not limited to, any diesel-electric hybrid vehicle, a gasoline-electric hybrid vehicle, a two-stroke engine, any and all burners or combustion units, including for example and without limitation herein, stationary burners (home heating, industrial, boilers, furnaces), waste incinerators, diesel fuel burners, diesel fuel engines (unit injected and common rail), jet engines, HCCI engines automotive diesel engines, gasoline fuel burners, gasoline fuel engines (PFI and DIG), power plant generators, and the like. The hydrocarbonaceous fuel combustion systems that may benefit from the present disclosure include all combustion units, systems, devices, and/or engines that burn fuels. By “combustion system” herein is also meant any and all internal and external combustion devices, machines, engines, turbine engines, jet engines, boilers, incinerators, evaporative burners, plasma burner systems, plasma arc, stationary burners, and the like which can combust or in which can be combusted a hydrocarbonaceous fuel.

The disclosed fuel compositions can be combusted in any combustion system, for example, an engine, such as a spark ignition engine or compression ignition engine, for example, advanced spark ignition and compression ignition engines with and without catalyzed exhaust after treatment systems with on-board diagnostic (“OBD”) monitoring. To improve performance, fuel economy and emissions, advanced spark ignition engines may be equipped with the following: direct injection gasoline (DIG), variable valve timing (VVT), external exhaust gas recirculation (EGR), internal EGR, turbocharging, variably geometry turbocharging, supercharging, turbocharging/supercharging, multi-hole injectors, cylinder deactivation, and high compression ratio. The DIG engines may have any of the above including spray-, wall-, and spray/wall-guided in-cylinder fuel/air charge aerodynamics. More advanced DIG engines in the pipeline will be of a high compression ratio turbocharged and/or supercharged and with piezo-injectors capable of precise multi-pulsing of the fuel into the cylinder during an injection event. Exhaust after treatment improvements will include a regeneratable NOₓ trap with appropriate operation electronics and/or a NOₓ catalyst. The advanced DIG engines described above will be use in gasoline-electric hybrid platforms.

In an aspect, there is disclosed a method for reducing deposit formation in a compression engine comprising providing to the engine a fuel composition comprising a deposit-modifying effective amount of a hydrocarbyl-substituted succinimide; a detergent; and a fuel. One of ordinary skill in the art would understand that “reducing deposit formation” is understood to be as compared to a vehicle utilizing an engine combusting a fuel composition that does not comprise a hydrocarbyl-substituted succinimide; a detergent, and a fuel. One of ordinary skill in the art would also understand that as deposit formation in a vehicle is thus reduced, then its fuel mileage, and/or fuel economy, is increased. This can be both from introduction of the present succinimide from the fuel into the engine, as well as the direct deposit-reducing effect of the succinimide on the piston and cylinder surfaces.

### EXAMPLES

Several fuel compositions were prepared by varying the type of additive and the amount of the additive present in the base fuel composition. The fuel compositions were then subjected to a modified ASTM D-6421 test with a Honda injector, wherein the amount of deposits on the new fuel injectors was measured as a percent of plugging. Four readings for each fuel composition were taken and the readings were averaged. The fuel compositions and results are shown in Table 1.

TABLE 1

| | | | Carrier fluid 2, | Dispersant, | Total solid | Dosage | lnj. | | Inj. | lnj. | |
|---|---|---|---|---|---|---|---|---|---|---|---|
| Ex. | Detergent, % | Carrier fluid 1, % | % | % | content, % | rate | 1 | lnj. 2 | 3 | 4 | Avg. |
| 1 | | | | | | Base fuel | 14.2 | 1.9 | 3.8 | 29.3 | 12.3 |
| 2 | H-6416 | AC-2400 | | | 54.40% | 80 ptb | 17.2 | 1.1 | 32.7 | 43.3 | 23.6 |
| | (30.22%) | (24.18%) | | | | | | | | | |
| 3 | | | | | | Base fuel | 11.7 | 16.6 | 0.8 | 13.8 | 10.7 |
| 4 | H-6416 | AC-2400 | | H-3192 | 47.10% | 80 ptb | 9.7 | 5.6 | 1.3 | 6.3 | 5.7 |
| | (29.05%) | (14.95%) | | (3.10%) | | | | | | | |
| 5 | H-6416 | X-105B | 500N (15.0%) | | 66.00% | 400 ppmv | 6.8 | 3.2 | 14.6 | 3.5 | 7 |
| | (30.40%) | (20.60%) | | | | | | | | | |
| 6 | | | | | | Base fuel | 7.9 | 1.9 | 22.9 | 18.5 | 12.8 |
| 7 | | | | | | Base fuel | 12.9 | 1.6 | 30.5 | 8.8 | 12.5 |
| 8 | H-6416 | X-105B | 500N (14.50) | H-3192 | 67.15% | 400 ppmv | 1.1 | 0.8 | 3 | 6 | 2.7 |
| | (29.38%) | (19.91%) | | (3.36%) | | | | | | | |

H-6416 is a detergent, commericial available from Afton Chemical
AC-2400 is a polyol, or Actaclear 2400, sold by Bayer
TPC-575 is a polyisobutylene (PIB) sold by Texas Petrochmical Corporation
X-105B, a polyol type carrier fluid sold in China
500N, a mineral oil carrier fluid sold in China
Column “Total Solid Content” is the percentage of solid in the formulation, excluding solvent.

As can be seen from the data, examples 4 and 8 which comprised the disclosed hydrocarbyl-substituted succinimide exhibited a reduced average amount of deposits as compared to the reference fuel compositions, which did not contain any additive, in examples 1, 3, 6, and 7. Moreover, example 4 and 8 exhibited a reduced average amount of deposits as compared to fuel compositions comprising other additives, such as the dibutylamine Mannich detergent in example 2.

It is noted that, as used in this specification and the appended claims, the singular forms “a,” “an,” and “the,” include plural referents unless expressly and unequivocally limited to one referent. Thus, for example, reference to “an antioxidant” includes two or more different antioxidants. As used herein, the term “include” and its grammatical variants are intended to be non-limiting, such that recitation of items in a list is not to the exclusion of other like items that can be substituted or added to the listed items

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

What is claimed is:

1. A method of making a hydrocarbyl-substituted succinimide comprising:
(i) reacting an olefinically unsaturated hydrocarbon with an anhydride to form a hydrocarbyl-substituted succinic anhydride; and
(ii) reacting the hydrocarbyl-substituted succinic anhydride with ammonia to form the hydrocarbyl-substituted succinimide
wherein the hydrocarbyl-substituted succinimide has an acidic proton.

2. The method of claim 1, wherein the olefinically unsaturated hydrocarbon comprises from about 10 to about 24 carbon atoms.

3. The method of claim 1, wherein the olefinically unsaturated hydrocarbon is linear or branched.

4. The method of claim 1, wherein the olefinically unsaturated hydrocarbon comprises from about 4 to about 36 carbon atoms.

5. The method of claim 1, wherein the olefinically unsaturated hydrocarbon comprises from about 14 to about 18 carbon atoms.

6. The method of claim 1, wherein the olefinically unsaturated hydrocarbon has a number average molecular weight ranging from about 100 to about 600.

7. The method of claim 1, wherein the olefinically unsaturated hydrocarbon is polyisobutylene.

8. The method of claim 1, wherein the anhydride is chosen from itaconic anhydride, citraconic anhydride, ethylmaleic anhydride, dimethylmaleic anhydride, and maleic anhydride.

9. The method of claim 1, wherein the steps of reacting are conducted at a temperature ranging from about 100° C. to about 250° C.

10. The method of claim 1, wherein a mole ratio of anhydride to olefinically unsaturated hydrocarbon is from about 5:1 to about 1:5.

11. The method of claim 1, wherein the step (i) is promoted by the inclusion of chlorine.

12. A method of making a hydrocarbyl-substituted succinimide consisting essentially of:
(i) reacting an olefinically unsaturated hydrocarbon with an anhydride to form a hydrocarbyl-substituted succinic anhydride; and
(ii) reacting the hydrocarbyl-substituted succinic anhydride with ammonia to form the hydrocarbyl-substituted succinimide
wherein the hydrocarbyl-substituted succinimide has an acidic proton.

13. A method of using a hydrocarbyl-substituted succinimide in a fuel composition comprising:
(i) reacting an olefinically unsaturated hydrocarbon with an anhydride to form a hydrocarbyl-substituted succinic anhydride; and
(ii) reacting the hydrocarbyl-substituted succinic anhydride with ammonia to form the hydrocarbyl-substituted succinimide; and
(iii) combining a fuel and the hydrocarbyl-substituted succinimide to make a fuel composition.

14. A fuel composition comprising:
a deposit-modifying effective amount of an ammonia-derived hydrocarbyl-substituted succinimide, wherein the hydrocarbyl-substituted succinimide has an acidic proton;
a detergent; and
a fuel.

15. The fuel composition of claim 14, wherein the detergent is selected from the group consisting of Mannich base detergents, polyetheramines, and hydrocarbyl amines.

16. The fuel composition of claim 14, wherein the detergent is present in an amount ranging from about 10 ppm to about 1500 ppm.

17. The fuel composition of claim 14, wherein the detergent is present in an amount ranging from about 100 ppm to about 300 ppm.

18. The fuel composition of claim 14, further comprising a carrier fluid.

19. The fuel composition of claim 14, further comprising at least one additive chosen from antioxidants, thermal stabilizers, carrier fluids, metal deactivators, dyes, markers, corrosion inhibitors, biocides, antistatic additives, drag reducing agents, demulsifiers, emulsifiers, dehazers, anti-icing additives, antiknock additives, anti-valve-seat recession additives, surfactants, other lubricity additives, combustion improvers, and cetane number improvers.

20. The fuel composition of claim 14, wherein the fuel is selected from the group consisting of diesel fuel, jet fuel, alcohols, ethers, kerosene, low sulfur fuels, synthetic fuels, coal, biomass, liquid petroleum gas, bunker oils, gas to liquid fuels, coal to liquid fuels, biomass to liquid fuels, high asphaltene fuels, fuels derived from coal, genetically engineered biofuels and crops and extracts therefrom, natural gas, propane, butane, unleaded motor and aviation gasolines, and so-called reformulated gasolines which contain both hydrocarbons of the gasoline boiling range and fuel-soluble oxygenated blending agents.

21. A method for reducing deposit formation in an engine comprising: providing to the engine a fuel composition comprising a deposit-modifying effective amount of an ammonia-derived hydrocarbyl-substituted succinimide; a detergent; and a fuel.
